(19) United States
(12) Patent Application Publication
(10) Pub. No.: US 2025/0297944 A1
(43) Pub. Date: Sep. 25, 2025

(54) METHOD FOR THE SPECTRAL TESTING OF SYSTEM COMPONENTS OF A MODULAR MEDICAL IMAGING SYSTEM

(71) Applicants: KARL STORZ SE & Co. KG, Tuttlingen (DE); DIASPECTIVE VISION GmbH, Am Salzhaff (DE)

(72) Inventors: Benedikt Köhler, Tuttlingen (DE); Hannes Köhler, Leipzig (DE)

(21) Appl. No.: 18/256,457

(22) PCT Filed: Dec. 7, 2021
(86) PCT No.: PCT/EP2021/084538

§ 371 Date: Jun. 17, 2025

(30) Foreign Application Priority Data
Dec. 9, 2020 (DE) 10 2020 132 818.4

(51) Int. Cl. G01N 21/27; A61B 1/00; A61B 1/04; G01N 21/64; H04N 17/00; H04N 23/10; H04N 23/50; H04N 23/88

(52) U.S. Cl. CPC G01N 21/27; G01N 21/6456; H04N 17/00; H04N 23/10; A61B 1/00057; A61B 1/00105; A61B 1/043; H04N 23/555; H04N 23/88

(57) ABSTRACT

A method for the spectral testing of system components is provided. The system components includes at least one optical component and at least one lighting component, which are designed, in a configuration intended for a function, to make up a modular medical imaging system, wherein in at least one measurement step, at least one test spectrum of a predefined test object is captured by means of the system components coupled to one another and a spectrometer, and, in at least one comparison step, the captured test spectrum is compared with at least one comparison spectrum characteristic of an intended function, wherein the imaging system is approved for further use if the test spectrum matches the comparison spectrum, or, if the test spectrum deviates from the comparison spectrum, a user is informed thereof before subsequent use of the imaging system, or the imaging system is blocked from subsequent use.

### CROSS-REFERENCE TO RELATED APPLICATIONS

This application is the U.S. national stage of PCT/EP2021/084538 filed on Dec. 7, 2021, which claims priority of German Patent Application No. DE 10 2020 132 818.4 filed on Dec. 9, 2020, the contents of which are incorporated herein.

### TECHNICAL FIELD

The disclosure relates to a method for the spectral testing of system components of a modular medical imaging system.

### BACKGROUND

Modular medical imaging systems are already known, which comprise system components, such as a light, an endoscope optical unit, and one camera or several thereof. Depending on the provided functionality, these can be combined with one another for white light imaging or also for fluorescence imaging. However, it is to be ensured that the system components configured for a respective functionality are used to make up the imaging system correctly. If an endoscope optical unit, which has an integrated fluorescence filter, is used, for example, as a system component for a functionality which is not provided, such as white light imaging, this results in a distorted representation. The use of a light configured for the functionality is also important. If a white light source is used, for example, in fluorescence imaging for the lighting, a superposition with the fluorescence signal to be actually detected can occur, due to which this background signal generated by the white light can be suppressed. To avoid the risk of incorrect use and combination of system components, providing them with a color code, with RFID chips, or the like is known.

### SUMMARY

The object of the disclosure is in particular to provide a generic device having improved properties with respect to security. The object is achieved according to the disclosure by the features of claim 1, while advantageous embodiments and refinements of the disclosure can be inferred from the dependent claims.

### Advantages of the Disclosure

The disclosure is directed to a method for the spectral testing of system components, specifically at least one optical component and at least one lighting component, which are configured in a configuration provided for a functionality to make up a modular medical imaging system.

It is proposed that, in at least one measurement step, at least one test spectrum of a specified test object is recorded by means of the system components coupled to one another and a spectrometer, and, in at least one comparison step, the recorded test spectrum is compared to at least one comparison spectrum characteristic of a provided functionality of the imaging device, and, if the test spectrum corresponds to the comparison spectrum, the imaging system is authorized for further use or, if the test spectrum deviates from the comparison spectrum, a user is notified thereof before subsequent use of the imaging system or the imaging system is blocked for subsequent use.

Operational reliability can advantageously be improved in this way, since incorrect use of system components for a functionality which is not provided is detectable.

Furthermore, damaged, defective, and/or counterfeit system components can be advantageously made detectable.

The method is in particular a test and/or calibration method, which is carried out chronologically before an examination of a patient by means of the medical imaging system. The method is in particular not carried out on a patient. A “modular medical imaging system” is to be understood in particular as a system which is assembled modularly from various exchangeable medical system components, which are configured for medical imaging. “Configured” is to be understood in particular as specially programmed, designed, provided, and/or equipped. An object being configured for a specific function is to be understood in particular to mean that the object fulfills and/or executes this specific function in at least one application and/or operating state. The medical imaging system is in particular an endoscopic, exoscopic, and/or microscopic imaging system. The optical component is in particular an optical unit, in particular such as an objective, an eyepiece, a relay optical unit, a filter optical unit of an endoscope, microscope, and/or an exoscope, in particular having fixed focal length and/or optical or digital zoom. The lighting component in particular comprises at least one light source, and preferably a light guide, which is configured to conduct light further from the light source. The light guide can be permanently connected to the light source here. Alternatively, the light guide can be detachably coupled to the light source. Alternatively, the light can also even be integrated distally in the endoscope. Functionalities of such an imaging system can be understood, depending on the configuration of the system components, as white light imaging, multispectral imaging (MSI) and/or hyperspectral imaging (HSI), fluorescence imaging, preferably for photodynamic diagnostics (PDD), or the like. A “configuration of the system components” is to be understood in particular as a combination and/or a sequence of the arrangement of these system components. In particular, the system components can differ from one another for various types of fluorescence imaging when various fluorescent pigments are used or in the case of autofluorescence or can be adapted to a special wavelength range of a respective fluorescence. For example, a light source of a lighting component can be adapted to the absorption spectrum of a fluorescent pigment. Furthermore, the optical components could be adapted to the emission spectrum of a fluorescent pigment. In the present method, the system components are coupled to the spectrometer and/or to one another in the measurement step. The system components are in particular connected upstream in the luminous flux of the spectrometer, so that a deviation of a test spectrum recorded by the spectrometer is influenced by the combination of the system components. Preferably, a test object is observed to record the test spectrum. The test object is in particular an object which consists at least largely of a homogeneous material composition and thus has advantageously macroscopically homogeneous spectral properties, such as a sheet of paper, a metal plate, or the like. Furthermore, the imaging system comprises at least one output unit, by means of which a user is notified of an incorrect configuration of the system components. The output unit can comprise an optical output element, such as a signal lamp, a display screen, or the like. Alternatively or additionally, the output unit could also have an acoustic output element, such as a loudspeaker. Haptic output elements would also be conceivable. Furthermore, action recommendations could be given to the user for how the problem can be remedied, for example by specifying which system components are to be exchanged.

Furthermore, the modular medical imaging system comprises at least one control unit, in which at least one operating program is stored and/or executable, which comprises at least the method for the spectral testing of system components of the modular medical imaging system. The control unit in particular comprises at least one processor. The processor is configured, for example, to execute the operating program. Furthermore, the control unit in particular comprises at least one memory. For example, the operating program is stored in the memory. The control unit is coupled to further system components of the imaging system, in order to activate them and/or output items of information, for example by means of the output unit.

It is proposed that the medical imaging system comprises further system components, specifically at least one further optical component, which is designed differently from the optical component, and/or a further lighting component, which is designed differently from the lighting component, which is/are combinable with the spectrometer instead of the optical component and/or the lighting component, wherein a configuration of the system components deviating from the provided configuration is detected in the comparison step. Operational reliability can advantageously be further improved, since it can be ensured on the basis of a test measurement whether the system components provided for a functionality are combined with one another.

It is proposed that, in the event of a detected deviation of the configurations of the system components from the provided configuration, it is proposed to the user which of the system components he has to exchange to obtain the provided configuration. Operational reliability can advantageously be further improved, since a solution for remedying a malfunction is automatically proposed to the user and he does not himself have to carry out an error diagnosis, which could in turn be flawed.

It is proposed that at least one provided configuration of the system components is configured for white light imaging, multispectral imaging, and/or hyperspectral imaging. The imaging system thus has an advantageous functionality by means of which a broad spectrum of medical analyses can be carried out, such as the detection of types of tissue and/or tissue properties, such as water content, fat content, oxygenation, deoxygenation, or the like.

It is proposed that at least one provided configuration of the system components is configured for fluorescence imaging; in this case, this is in particular a further provided configuration which is different from the preceding configuration. The imaging system thus has an advantageous functionality by means of which a broad spectrum of medical analyses can be carried out, such as perfusion analysis, tumor detection, or the like. The various provided configurations differ here in particular by way of a use and/or arrangement of the system components. Fluorescence imaging in particular involves fluorescence of a fluorescent pigment administered to the tissue, such as indocyanine green, fluorescein, 5-aminolevulinic acid (5-ALA), autofluorescence, or the like.

It is proposed that the imaging system has a multispectral and/or hyperspectral camera, which is configured to record at least one multispectral and/or hyperspectral image and which includes the spectrometer. Additional components can advantageously be omitted, since the spectrometer is already part of the multispectral and/or hyperspectral camera.

It is proposed that the test spectrum is taken from the multispectral and/or hyperspectral test image. A particularly rapid test step can advantageously be carried out, since a recording of the test spectrum by means of only a single pixel, which is recorded using the multispectral and/or hyperspectral camera, can be used. To increase an accuracy of the test spectrum and reduce background noise, however, it is also conceivable that averaging can be carried out over multiple pixels, rows, columns and/or an entire test image recorded using the multispectral and/or hyperspectral camera in order to determine the test spectrum.

It is proposed that the comparison step is carried out simultaneously with a white balance of the imaging system. A configuration time of the imaging system can advantageously be shortened.

It is proposed that the modular medical imaging system comprises at least one endoscope, one exoscope, and/or one microscope. A diverse use of the imaging system can advantageously be achieved.

Further advantages result from the following description of the drawings. An exemplary embodiment of the disclosure is shown in the drawings. The drawings, the description, and the claims contain numerous features in configuration. A person skilled in the art will expediently also consider the features individually and combine them to form reasonable further configurations.

### BRIEF DESCRIPTION OF DRAWINGS

FIG. 1 shows a schematic illustration of an imaging system having system components in a perspective view,

FIG. 2 shows a schematic illustration of a lighting spectrum of the lighting component of the imaging system,

FIG. 3 shows a schematic illustration of spectral properties characteristic of indocyanine green and of further lighting spectra of a further lighting component of the imaging system and further optical components of the imaging system,

FIG. 4 shows a schematic illustration of a camera of the imaging system in a top view,

FIG. 5 shows a schematic flow chart of an exemplary operating method of the imaging system,

FIG. 6 shows a schematic diagram having a test spectrum of the test object recorded using the imaging system in the test step, and a comparison spectrum.

### DETAILED DESCRIPTION OF PREFERRED EMBODIMENTS

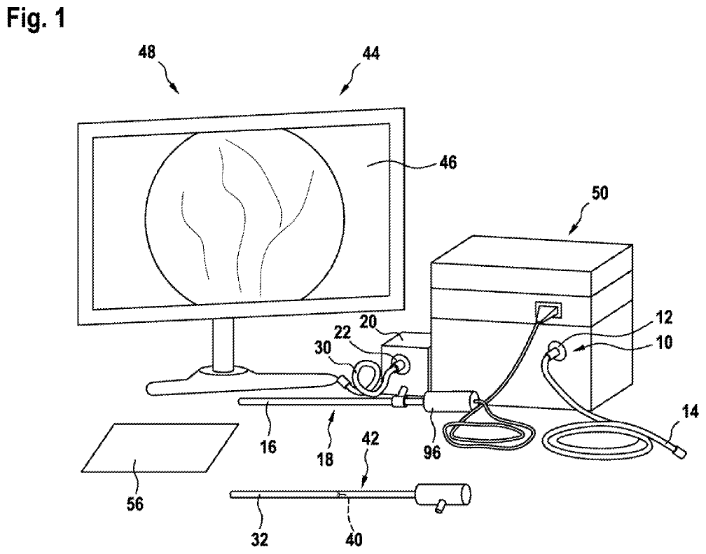

FIG. 1 shows a schematic illustration of a modular medical imaging system in a perspective view. The imaging system comprises multiple system components.

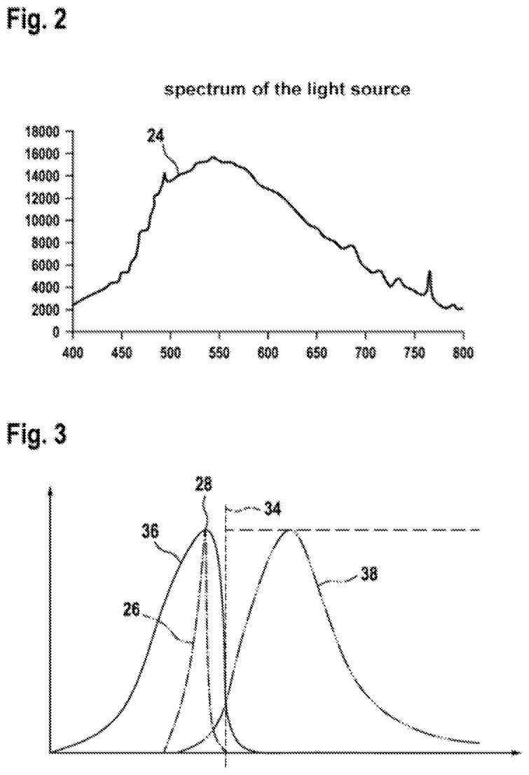

The imaging system comprises as a first system component a lighting component 10. The lighting component 10 is configured for lighting an examination area. The lighting component 10 comprises at least one light source 12. In the present case, the light source 12 is a white light source such as a homogenized xenon lamp, a phosphor-modified LED, or the like. A light source spectrum 24 generated by the light source 12 is shown in FIG. 2. Furthermore, the lighting component 10 comprises a light guide 14. The light guide 14 is connected to the light source 12. The light guide can be, for example, a bundle of optical fibers.

Furthermore, the imaging system comprises as a second system component an optical component 16. The optical component 16 is designed in the present case as an endoscope optical unit. The optical component 16 comprises at least one objective. Furthermore, the optical component 16 can comprise various filters, for example, for filtering various fluorescence wavelengths or also a relay optical unit for relaying optical images, and an optical zoom device and/or focus device.

The imaging system comprises an endoscope 18. The optical component 16 is integrated in the endoscope 18. Furthermore, the lighting component 10 is connected to the endoscope 18. Instead of an endoscope 18, the imaging system could also have an exoscope and/or a microscope, however.

The lighting component 10 and the optical component 16 are configured for a provided functionality. In the present case, the lighting component 10 and the optical component 16 are configured for white light imaging, multispectral imaging, and/or hyperspectral imaging.

The imaging system comprises as a further first system component a further lighting component 20. The further lighting component 20 is configured for lighting an examination area. The further lighting component 20 comprises at least one further light source 22. The further light source 22 is designed differently from the light source 12. A light source spectrum 26 generated by the further light source 22 is shown in FIG. 3. In the present case, the further light source 22 is an LED which has an intensity maximum in the range of an absorption maxima 28 of a fluorescent pigment. The fluorescent pigment can be, for example, indocyanine green. Furthermore, the further lighting component 20 comprises a further light guide 30. The light guide 30 is adapted to the lighting spectrum 28 of the further light source 22. The further light guide 30 is connected to the further light source 22. The further light guide 30 can be, for example, a bundle of optical fibers.

Furthermore, the imaging system comprises as a further second system component a further optical component 32. The further optical component 32 is designed in the present case as a further endoscope optical unit. The further optical component 32 comprises at least one objective. Furthermore, the further optical component 32 comprises a filter 40, which is adapted to the absorption spectrum 36 or emission spectra 38 of the fluorescent pigment used. The filter 40 is designed in the present case as an edge filter, the filter edge 34 of which lies in the middle between the absorption spectrum 36 and the emission spectrum 38 of the fluorescent pigment. FIG. 3 shows the filter edge 34 of the filter. In this way, the filter 40 blocks light which originates, for example, from the further lighting component 20. However, the filter 40 is transmissive for fluorescent light of the fluorescent pigment. The filter is also at least partially transmissive for the lighting spectrum 24 of the lighting component (cf. FIG. 2).

The imaging system comprises a further endoscope 42. The further optical component 32 is integrated in the further endoscope 42. Furthermore, the further lighting component 20 is connectable or connected to the further endoscope 42. Instead of a further endoscope, however, the imaging system could also have a further exoscope and/or microscope.

The further lighting component 20 and the further optical component 32 are configured for a specific functionality. In the present case, the further lighting component 20 and the further optical component 32 are configured for fluorescence imaging.

Furthermore, the imaging system includes at least one camera 96. The camera 96 is designed as a multispectral and/or hyperspectral camera. The camera 96 is arranged or arrangeable proximally on the endoscope 18 or the further endoscope 42. The camera 96 includes a camera housing 168. Further components of the camera 96 are arranged in the camera housing 168.

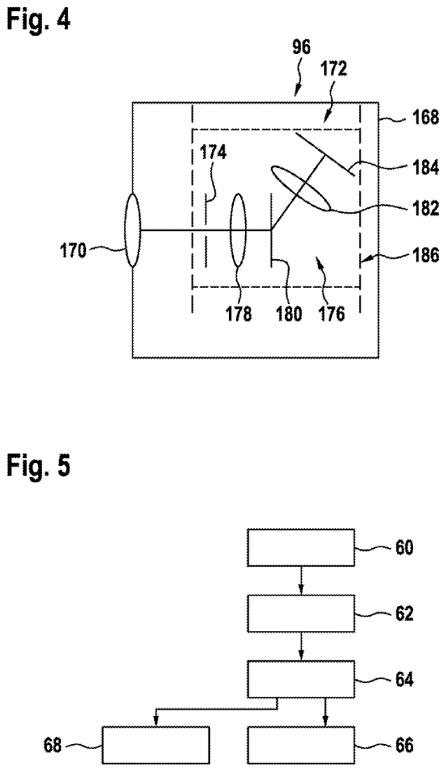

FIG. 4 shows a structure of the camera 96 in a schematic illustration. The camera 96 includes at least one input objective 170. The input objective 170 is arranged in the camera housing 168.

The camera 96 includes a spectrometer 172. The spectrometer 172 is connected to the control unit 102 for activation. The spectrometer 172 is arranged in the camera housing 168. The spectrometer 172 is arranged upstream in the luminous flux behind the input objective 170.

The spectrometer 172 includes at least one aperture 174. The input objective 170 focuses 170 the image on the aperture 174. The aperture 174 is arranged in an image plane of the image generated by the input objective 170. A distance of the input objective 170 and the aperture 174 corresponds at least essentially to the image distance of the input objective 170. The aperture 174 lies in the image plane. The aperture 174 is configured to select an area of the image generated by the input objective 170. For this purpose, the aperture 174 includes an opening. The opening has the shape of a slit. A main extension direction of the opening defines a first direction. This first direction is at least essentially parallel to the image plane of the image generated by the input objective 170. The aperture 174 is configured to select a strip of the image which has a width of at least 15 μm and/or of at most 30 μm.

The spectrometer 172 includes an internal optical unit 176. The internal optical unit 176 is arranged upstream in the luminous flux behind the aperture 174. The internal optical unit 176 includes at least one internal lens 178. This internal lens 178 is arranged upstream in the luminous flux behind the aperture 174. A distance of the internal lens 178 to the aperture 174 corresponds to the focal length of the internal lens 178. In this way, the internal lens 178 images the aperture 174 in infinity.

Furthermore, the spectrometer 172 includes at least one dispersive element 180. The dispersive element 180 is arranged upstream in the luminous flux behind the internal lens 178. The dispersive element 180 is configured for a wavelength-dependent dispersion of light. In the present case, the dispersive element 180 is configured to disperse this light in a second direction. The second direction is at least essentially perpendicular to the main extension of the opening of the aperture. For example, the dispersive element can be a prism. In the present case, the dispersive element 180 is an optical grating, in particular designed as a blaze grating.

The internal optical unit 176 includes at least one further internal lens 182. The further internal lens 182 is arranged upstream in the luminous flux behind the dispersive element 180. In this way, the dispersive element 180 is arranged between internal lens 178 and the further internal lens 182. In other words, the dispersive element 180 is arranged inside the internal optical unit 176. A distance of the further internal lens 182 to the dispersive element 180 corresponds to the focal length of the further internal lens 182. The further internal lens 182 is configured to sharply image the light dispersed by the dispersive element 180.

The spectrometer 172 includes a camera sensor 184. The camera sensor 184

is connected to the control unit 102. The camera sensor 184 is arranged upstream in the luminous flux behind the further internal lens 182. In other words, the further internal lens 182 is arranged between the dispersive element 180 and the camera sensor 184. The camera sensor 184 is a monochromatic sensor. Such a monochromatic sensor only has a single spectral sensitivity. The camera sensor 184 is a two-dimensional CMOS digital camera sensor. Alternatively, it could be a CCD digital camera sensor.

The camera 96 includes an adjustment unit 186. The adjustment unit 186 is connected for control to the control unit 102. The adjustment unit 186 is arranged in the camera housing 168. The adjustment unit 186 is configured to adjust at least the aperture 174 in relation to the input objective 170. In the present case, the entire spectrometer 172 is adjusted relative to the input objective 170. The adjustment unit 186 includes at least one bearing. The bearing is configured for a movable mounting of the spectrometer relative to the input objective. In the present case, the bearing is designed as a linear bearing. For example, the bearing can comprise guide rails, which are arranged extending along the second direction. The adjustment unit 186 furthermore includes an adjustment actuator for the drive. The adjustment actuator is designed in the present case as a linear actuator. To achieve a uniform adjustment, for example, the adjustment actuator could be designed as a piezoelectric actuator.

By adjusting the aperture relative to the input objective, spectra can be recorded for various image details of the examination area to be examined. The entire examination area can thus be spectrally scanned by displacement, due to which an image including spectral information may be generated.

Furthermore, the imaging system includes an output unit 44. In the present case, the output unit 44 comprises at least one output element 46. The output element 46 is an optical output element. The output element 46 is designed as a display screen. Alternatively, a mobile terminal can also be used as the output element, such as a tablet, a smartphone, or the like. The output unit 44 is configured to output information of the imaging system. For example, images recorded using the imaging system are displayable on the output element 46.

Furthermore, the imaging system comprises at least one input unit 48. The input unit 48 can be, for example, a keyboard. In the present case, however, it is a touchscreen, which is also part of the display screen of the display unit 44.

The imaging system comprises a control unit 50. The control unit 50 is configured for control of further components of the imaging system and is connected thereto. The control unit 50 comprises a memory. An operating program is stored in the memory. Furthermore, the control unit includes a processor. The operating program is executable by the processor.

FIG. 5 shows a schematic flow chart of an exemplary method for testing and/or calibrating the imaging system. The method is part of the operating program.

The method comprises at least one method step 60. In the method step 60, a user inputs a provided functionality of the imaging system. For this purpose, the user uses the input unit 48. For example, he selects white light imaging as the provided functionality.

The method comprises a further method step 62. In the further method step 62, the user selects system components and connects them to one another, so that they are in a fixed configuration. For example, the system components configured for the previously selected provided functionality could be proposed to the user on the output unit.

The method comprises a measurement step 64. In the measurement step 64, at least one test spectrum 58 of a provided test object 56 is recorded by means of the system components coupled to one another and a spectrometer 172. The test object 56 is a sheet of paper in the present case. However, for this purpose an image of the test object 56 does not have to be generated or evaluated by means of the camera 96, rather it is sufficient to record a single line or pixel of an image of the test object 56 by means of the spectrometer 172.

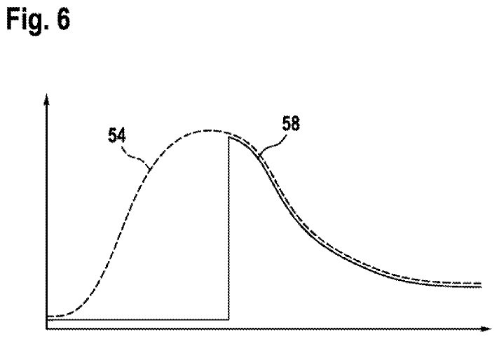

The method comprises at least one comparison step 66. In the comparison step 66, the test spectrum 58 is compared to at least one comparison spectrum 54 characteristic of the previously selected provided functionality of the imaging device. An exemplary diagram of such a test spectrum 58 and such a comparison spectrum 54 is shown in FIG. 6. If the test spectrum 58 corresponds to the comparison spectrum 54, the imaging system is authorized for further use. In the present case, a deviation of the test spectrum 58 from the comparison spectrum 54 may be seen in FIG. 6. Specifically, a flank can be seen which can be assigned to the filter, which is actually configured for fluorescence imaging. It can therefore be concluded that at least the correct optical component was not used for the provided functionality. The comparison step 66 is carried out simultaneously with a white balance step 68, in which a white balance of the imaging system takes place.

If the spectrum deviates from the comparison spectrum, the user is notified thereof before subsequent use of the imaging system. For this purpose, a corresponding warning is output on the display unit 44. Alternatively or additionally, the imaging system is blocked for subsequent use. The lighting source can be deactivated for this purpose, for example. Furthermore, it is proposed to the user which of the system components he has to exchange in order to obtain the provided configuration. In the present case, it is proposed to the user that he exchange the optical system component 32, since it has a filter 40 which is not suitable for the provided functionality.

1. A method for the spectral testing of system components, specifically at least one optical component and at least one lighting component of the system components, which are configured in a configuration provided for a functionality to make up a modular medical imaging system, wherein, in at least one measurement step, at least one test spectrum of a specified test object is recorded by means of the system components coupled to one another and a spectrometer, and, in at least one comparison step, the recorded test spectrum is compared to at least one comparison spectrum characteristic of a provided functionality, wherein, when the test spectrum corresponds to the comparison spectrum, the imaging system is authorized for further use or, when the test spectrum deviates from the comparison spectrum, a user is notified thereof before subsequent use of the imaging system or the imaging system is blocked for subsequent use.

2. The method as set forth in claim 1, wherein the system components further includes at least one further optical component which is designed differently from the optical component, and/or a further lighting component which is designed differently from the lighting component, the further lighting component and the further optical component being combinable with the spectrometer instead of the optical component and/or the lighting component, wherein a configuration of the system components deviating from the provided configuration is detected in the comparison step.

3. The method as set forth in claim 1, wherein, in the event of a detected deviation of the configurations of the system components from the provided configuration, it is proposed to the user which of the system components are to be exchanged to obtain the provided configuration.

4. The method as set forth in claim 1, wherein at least one provided configuration of the system components is configured for white light imaging, multispectral imaging, and/or hyperspectral imaging.

5. The method as set forth in claim 1, wherein at least one provided configuration of the system components is configured for fluorescence imaging.

6. The method as set forth in claim 1, wherein a multispectral and/or hyperspectral camera, which is configured to record at least one multispectral and/or hyperspectral image, is used as the spectrometer.

7. The method as set forth in claim 6, wherein the test spectrum is taken from the multispectral and/or hyperspectral image.

8. The method as set forth in claim 1, wherein the comparison step is carried out simultaneously with a white balance of the imaging system.

9. The method as set forth in claim 1, wherein the modular medical imaging system comprises at least one endoscope, one exoscope, and/or one microscope.

10. A modular medical imaging system having at least one control unit, in which at least one operating program is stored and/or executable, which the method for the spectral testing of system components of the modular medical imaging system as set forth in claim 1.
